(19) United States
(12) Patent Application Publication
(10) Pub. No.: US 2011/0166360 A1
(43) Pub. Date: Jul. 7, 2011

(54) Catalytic Process for Asymmetric Hydrogenation

(75) Inventors: Alexander Beliaev, Mindelo (PT); David Alexander Learmonth, Alfena (PT); Juan José Almena Perea, Hanau (DE); Gerhard Geib, Hofheim (DE); Patrick Hitzel, Munzenberg (DE); Renat Kadyrov, Frankfurt (DE); David Voigtlaender, Bad Vilbel (DE)

(73) Assignee: BIAL - PORTELA & CA, S.A., S. Mamede do Coronado (PT)

(21) Appl. No.: 12/921,961

(22) PCT Filed: Mar. 13, 2009
(86) PCT No.: PCT/PT09/00012

§ 371 Date: Mar. 28, 2011

(30) Foreign Application Priority Data
Mar. 13, 2008 (US) 61/036121

(51) Int. Cl. C07D 405/04; C07D 311/04

(52) U.S. Cl. 548/311.4; 549/404

(57) ABSTRACT

A process for preparing the S or R enantiomer of a compound of formula A,

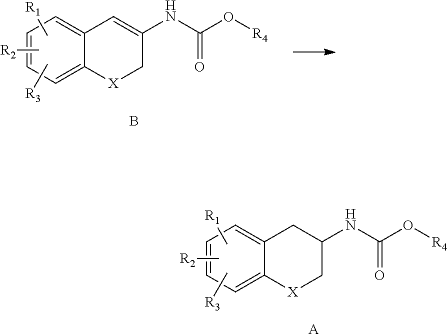

the process comprising subjecting a compound of formula B to asymmetric hydrogenation in the presence of a chiral transition metal catalyst and a source of hydrogen, wherein: X is CH₂, oxygen or sulphur; R₁, R₂ and R₃ are the same or different and signify hydrogen, halogen, alkyl, alkyloxy, hydroxy, nitro, alkylcarbonylamino, alkylamino or dialkylamino group; and R₄ is alkyl or aryl, the transition metal catalyst comprising a chiral ligand having the formula

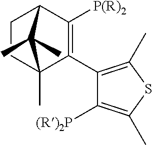

wherein each R and R′ independently represents alkyl, aryl, aralkyl, alkenyl, alkynyl, alkoxy, aryloxy, alkylthio, arylthio, unsubstituted or substituted cyclic moiety selected from a group consisting of monocyclic or polycyclic saturated or partially saturated carbocyclic or heterocyclic, aromatic or heteroaromatic rings said rings comprising from 4 to 8 atoms and comprising from 0 to 3 heteroatoms, wherein: the term alkyl means hydrocarbon chains, straight or branched, containing from one to six carbon atoms, optionally substituted by aryl, alkoxy, halogen, alkoxycarbonyl or hydroxycarbonyl groups; the term aryl means an aromatic or heteraromatic group, optionally substituted one or more times by alkyl, alkyloxy, halogen or nitro group; and the term halogen means fluorine, chlorine, bromine or iodine.

### CROSS-REFERENCE TO RELATED APPLICATIONS

This application is a filing under 35 U.S.C. 371 of International Application No. PCT/PT2009/000012 filed Mar. 13, 2009, entitled “Catalytic Process for Asymmetric Hydrogenation,” which is a non-provisional of and claims priority to U.S. Provisional Patent Application No. 61/036,121 filed on Mar. 13, 2008, which applications are incorporated by reference herein in their entirety.

### BACKGROUND OF THE INVENTION

The present invention relates to an improved catalytic process for asymmetric hydrogenation. In particular, the present invention relates to a process for preparing intermediates useful in the synthesis of peripherally-selective inhibitors of dopamine-(β-hydroxylase (DβH), the process involving catalytic asymmetric hydrogenation and to advantageous ligands, and novel catalysts incorporating the ligands, for use in the hydrogenation.

(R)-5-(2-Amino ethyl)-1-(6,8-difluoro chroman-3-yl)-1,3-dihydroimidazole-2-thione hydrochloride (the compound of formula 1, below) is a potent, non-toxic and peripherally selective inhibitor of DβH, which can be used for treatment of certain cardiovascular disorders. Compound 1 is disclosed in WO 2004/033447, along with processes for its preparation.

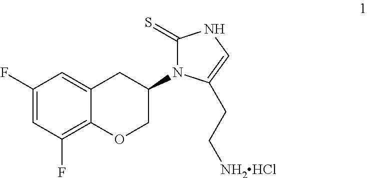

The process disclosed in WO 2004/033447 involves the reaction of (R)-6,8-difluorochroman-3-ylamine hydrochloride, [4-(tert-butyldimethylsilanyloxy)-3-oxobutyl]carbamic acid tert-butyl ester and potassium thiocyanate. The structure of (R)-6,8-difluorochroman-3-ylamine is shown below as compound 2:

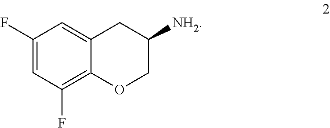

(R)-6,8-difluorochroman-3-ylamine (compound 2) is a key intermediate in the synthesis of compound 1. The stereochemistry at the carbon atom to which the amine is attached gives rise to the stereochemistry of compound 1, so it is advantageous that compound 2 is present in as enantiomerically pure a form as possible. In other words, the desired (e.g., R) enantiomer should be in predominance, with little or none of the undesired (e.g., S) enantiomer present. Thus, advantageously the R-enantiomer, shown above as compound 2, is produced with as high an enantiomeric excess as possible.

### BRIEF SUMMARY OF THE INVENTION

An advantageous process for preparing a precursor of, for example, the compound of formula 2 has now been found. The process involves catalytic asymmetric hydrogenation of a corresponding ene-carbamate using a transition metal catalyst comprising a chiral ligand having the formula:

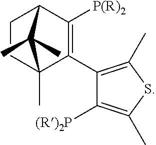

Such ligands and processes for their production are described in EP 1595888A1. The process may also be employed in the preparation of similar precursors useful in the production of other peripherally-selective inhibitors of dopamine-β-hydroxylase. The catalyst is particularly advantageous as it shows high activity and selectivity in the asymmetric hydrogenation reaction. Levels of activity and selectivity have also been shown to be improved when the hydrogenation is carried out in the presence of acid additives. Furthermore, the catalysts have been shown to be highly effective when hydrogenation is carried out on a large scale, which makes the catalysts highly suitable for industrial use. More specifically, it has been found that, with 800 g substrate, the desired chiral product may be produced with optical purity greater than 99% and at a yield over 90%.

### DETAILED DESCRIPTION OF THE INVENTION

According to a first aspect of the present invention, there is provided a process for preparing the S or R enantiomer of a compound of formula A,

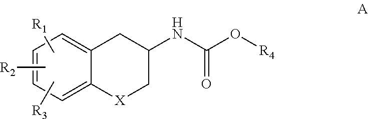

the process comprising subjecting a compound of formula B to asymmetric hydrogenation in the presence of a chiral transition metal catalyst and a source of hydrogen,

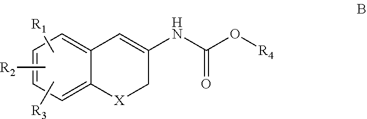

wherein X is CH₂, oxygen or sulphur; R₁, R₂, and R₃ are the same or different and signify hydrogens, halogens, alkyl, alkyloxy, hydroxy, nitro, alkylcarbonylamino, alkylamino, or dialkylamino group; and R₄ is alkyl or aryl, the transition metal catalyst comprising a chiral ligand having the formula:

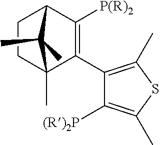

wherein each R or R′ group independently represents alkyl, aryl, aralkyl, alkenyl, alkynyl, alkoxy, aryloxy, alkylthio, arylthio, unsubstituted or substituted cyclic moiety selected from the group consisting of monocyclic or polycyclic saturated or partially saturated carbocyclic or heterocyclic, or aromatic or heteraromatic rings, said rings comprising from 4 to 8 atoms and optionally comprising from 1 to 3 heteroatoms, and wherein the term alkyl, whether alone or in combination with other moieties, means hydrocarbon chains, straight or branched, containing from one to six carbon atoms, optionally substituted by aryl, alkoxy, halogen, alkoxycarbonyl or hydroxycarbonyl groups, the substituents themselves optionally being substituted; the term aryl means an aromatic or heteraromatic group optionally substituted by alkyloxy, halogen or nitro group; and the term halogen means fluorine, chlorine, bromine or iodine. The substituents may themselves be substituted. In an embodiment, the term aryl may mean an aromatic ring comprising from 4 to 8 atoms and optionally comprising from 1 to 3 heteroatoms. Suitably, aryl means phenyl or naphthyl. Compound B may be referred to as an ene-carbamate.

The chiral ligands used in the process of the present invention are from a series of ligands known under the trade name “catASium™ T”. Throughout this specification, references to the “catASium™ T” series of ligands refers to the chiral ligands having the formula:

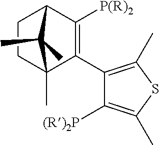

In an embodiment, the source of hydrogen is hydrogen gas.

In an embodiment, X is O. In another embodiment, at least one of R₁, R₂, and R₃ is halogen, preferably fluorine. Preferably, two of R₁, R₂, and R₃ are halogen, preferably fluorine, and the other of R₁, R₂, and R₃ is hydrogen. Suitably, compound A has the following formula:

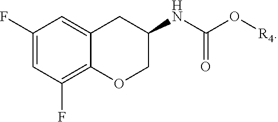

In an embodiment, R₄ is C₁ to C₄ alkyl. Optionally, R₄ is methyl (i.e., the methyl-substituted ene-carbamate), ethyl (i.e., the ethyl-substituted ene-carbamate) or ^{t}Bu (i.e., the ^{t}Bu-substituted ene-carbamate). Preferably, R₄ is methyl. In an alternative embodiment, R₄ is benzyl (i.e., the benzyl-substituted ene-carbamate).

Preferably, the transition metal in the catalyst is rhodium or ruthenium. Most preferred is ruthenium.

Ruthenium-catalysed hydrogenation investigations have revealed that full conversion and ee's more than 90% and up to 95% were obtained using the methyl-substituted ene-carbamate in the presence of catASium™ T series-based catalysts.

Asymmetric hydrogenation using a rhodium-based catalyst has also been investigated. In particular, [Rh-(catASium™)(L)]X″ cationic complexes (where L=cyclooctadiene, and X″=BF₄) have been investigated. Rh-catASium®-catalysed hydrogenation revealed moderate to high activity and low enantioselectivity for the ene-carbamate substrates.

Suitably, the catalyst has the formula [(catASium™ T)Ru(arene)X′]Y, [(catASium™ T)Ru(L)₂] or [(catASium™ T)Ru(L′)₂X′₂], wherein X′ is a singly-negative monodentate ligand, Y is a balancing anion, L is a monovalent negative coordinating ligand, and L′ is a non-ionic monodentate ligand.

In an embodiment, X′ is chloride. In another embodiment, Y is chloride. Both X′ and Y may be chloride. In another embodiment, arene is p-cymene or benzene. Preferably, L is acac. Suitably, L′ is dimethylformamide (dmf). Other options for the ligand include acetyl, trifluoroacetyl, tetrafluoroborate, and mono- and diamines.

Alternatively, the catalyst is Ru(catASium™ T ligand)(acac)₂, Ru(catASium™ T ligand)Br₂, Ru(catASium™ T ligand)Cl₂(Ar) wherein Ar is C₆H₆ (i.e., benzene) or p-cymene, or Ru(catASium™ T ligand)Cl₂(dmf)ₓ, wherein x is suitably 2, 3, or 4. Suitable examples of ligands from the T series are shown in Scheme 1 below. Ligands having the opposite stereochemistry to that of the ligands in Scheme 1 may also be used in the asymmetric hydrogenation of the present invention.

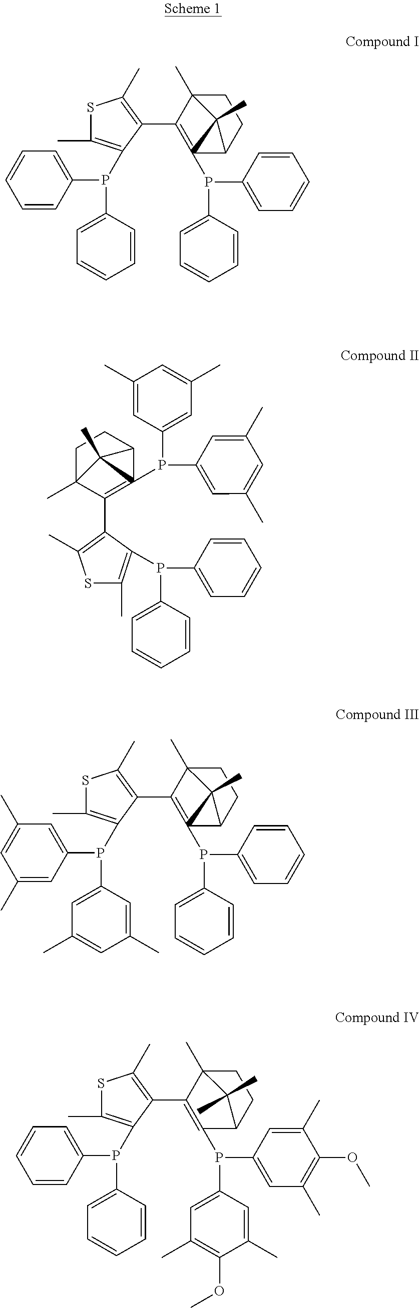

Compound I is known by the trade name catASium™ T1. Compound II is known by the trade name catASium™ T2. Compound III is known by the trade name catASium™ T3. Compound IV is known by the trade name catASium™ T4. Throughout this specification, references to catASium™ T1, T2, T3, or T4 refer to compounds I, II, III, or IV, respectively, having the respective structures shown above.

Preferably, the ligand is the R or S enantiomer of catASium™ T3. catASium™ T3 has the chemical name (1R)-3-diphenylphosphino-[4-di-(3,5-dimethylphenyl)phosphino-2,5-dimethylthienyl-3)-1,7,7-trimethylbicyclo[2.2.1]heptene-2. Suitably, the ligand is the R enantiomer of catASium™ T3.

Preferably, the active transition metal catalysts are pre-formed prior to the hydrogenation reaction. Alternatively, the active transition metal catalysts are formed in situ, i.e., the catalyst is not isolated prior to the hydrogenation reaction, but is formed from its precursor ligands in the reaction pot. The catalysts may have been pre-formed from precursor compounds. For example, Ru(catASium™ T ligand)(acac)₂ may have been prepared from Ru(η-4-hexadien)(acac)₂ and the catASium™ T ligand. Ru(catASium™ T ligand)Br₂ may have been prepared from Ru(methylallyl)₂COD, the catASium™ T ligand and HBr. The Ru(catASium™ T ligand)Cl₂(C₆H₆) may have been prepared from [Ru(C₆H₆)Cl₂]₂, the catASium™ T ligand and a 1:1 mixture of dichloromethane/ethanol. The Ru(catASium™ T ligand)Cl₂(p-cymene) may have been prepared from [Ru(p-cymene)Cl₂]₂, the catASium™ T ligand and a 1:1 mixture of dichloromethane/ethanol. Ru(catASium™ T ligand)Cl₂(dmf)ₓ may have been prepared from [Ru(C₆H₆)Cl₂]₂, the catASium™ T ligand and DMF.

Preferably, the substrate:catalyst (S/C) ratio is from 100/1 to 5000/1, more preferably from 250/1 to 4000/1, still more preferably from 500/1 to 2000/1. Yet more preferably from 1000/1 to 2000/1. Most preferably, the S/C ratio is 2000/1.

Preferably, the hydrogenation is conducted at a temperature ranging from 40° C. to 100° C., more preferably at a temperature ranging from 40° C. to 90° C., more preferably still at a temperature ranging from 50° C. to 90° C., even more preferably at a temperature ranging from 60° C. to 90° C., and most preferably, the hydrogenation is carried out at a temperature of 80° C.

Preferably, the hydrogenation is carried out at a pressure ranging from 10 bars to 70 bars, more preferably at a pressure ranging from 10 bars to 60 bars, even more preferably at a pressure ranging from 20 bars to 50 bars, even more preferably still at a pressure ranging from 20 bars to 40 bars, and yet still more preferably at a pressure ranging from 20 bars to 30 bars. Most preferably, the hydrogenation is carried out at a pressure of 20 or 30 bars.

In a most preferred embodiment, the hydrogenation is carried out in the presence of an acid. Suitable acids include HBF₄, HCl, HBr, H₂SO₄, CF₃SO₃H, CH₃COOH and H₃PO₄. Preferably the acid is a weak acid, such as ethanoic acid or phosphoric acid. Suitably, ethanoic acid is present in concentrations ranging from 50% (v/v) to 20% (v/v). Phosphoric acid may be present in concentrations from 10% (v/v) to 0.01% (v/v), preferably 5% (v/v) to 0.01% (v/v), more preferably 1% (v/v) to 0.01% (v/v), still more preferably 0.5% (v/v) to 0.05% (v/v). The most preferred concentration of phosphoric acid is 0.1% (v/v).

In an embodiment, the acid is present in a solvent. For example, the acid solvent is diethyl ether or water. The concentration of the acid solution is typically 80% (w/w) to 90% (w/w), preferably 85% (w/w). The most preferred phosphoric acid solution is 85% (w/w) in water.

The hydrogenation is preferably conducted in a solvent. The solvent may be selected from a substituted or unsubstituted straight- or branched-chain C1 to C6 alcohol, an arene or mixtures thereof. Suitable solvents include MeOH, EtOH, i-PrOH, 1-PrOH, 1-BuOH, 2-BuOH, CF₃CH₂OH, dichloromethane (DCM), dichloroethane (DCE), tetrahydrofuran (THF), toluene or a 1:1 mixture of MeOH and DCM. The solvent is preferably MeOH or DCM. Most preferably, the solvent is MeOH.

Preferably, the reaction mixture is mixed thoroughly throughout the hydrogenation process.

In a further embodiment, the process further comprises subsequently crystallising the compound of formula A. Optionally, the crystallisation is carried out in DCM/hexane.

In an embodiment, compound A is in the form of the S enantiomer. In an alternative embodiment, compound A is in the form of the R enantiomer.

Compound B may be prepared, for example, by the process described in Tetrahedron: Asymmetry 10 (1999) 3467-3471.

In a still further embodiment, the process further comprises converting the R or S enantiomer of compound A to the respective R or S enantiomer of a compound of formula C, or a salt thereof

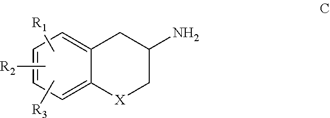

The compound A may be converted to compound C by a reaction involving substituting the group —C(═O)—O—R₄ with H.

In an embodiment, the R or S enantiomer of compound A is converted to the respective R or S enantiomer of the compound of formula C by hydrolysis. Hydrolysis may be carried out using 40% potassium hydroxide in methanol, followed by isolation of the crude amine and crystallisation of the amine as a salt with L-tartaric acid.

In another aspect of the present invention, there is provided a process for forming the R or S enantiomer of a compound of formula E or a salt thereof:

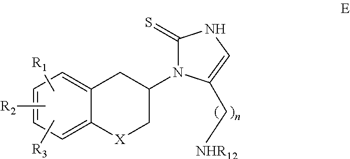

comprising forming the R or S enantiomer of a compound of formula C according to the process described above, and converting the R or S enantiomer of the compound of formula C to the R or S enantiomer of the compound of formula E. In an embodiment, compound C is converted to the compound E by using the compound C as an amino component to build the N(1) moiety of the substituted imidazole-2-thione ring of compound E. In an embodiment, the amino group on the compound C is converted to a 5-substituted imidazole-2-thione group, wherein the substituent at position 5 is the group —(CH₂)ₙ—NHR₁₂, wherein R₁₂ signifies hydrogen, alkyl, or alkylaryl group.

In a yet further embodiment, the process further comprises reacting the R or S enantiomer of the compound of formula C with a compound of formula D

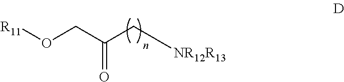

where n signifies 1, 2 or 3; when n is 1 or 2, R₁₂ signifies hydrogen, alkyl, or alkylaryl group, R₁₁ signifies a hydroxyl protecting group and R₁₃ signifies an amino protecting group; when n signifies 3, R₁₁ signifies a hydroxyl protecting group but R₁₂ and R₁₃ taken together represent a phthalimido group; with a water soluble thiocyanate salt in the presence of an organic acid in a substantially inert solvent, wherein the water soluble thiocyanate salt is an alkali metal thiocyanate salt or a tetraalkylammonium thiocyanate salt, to produce intermediate products E to H

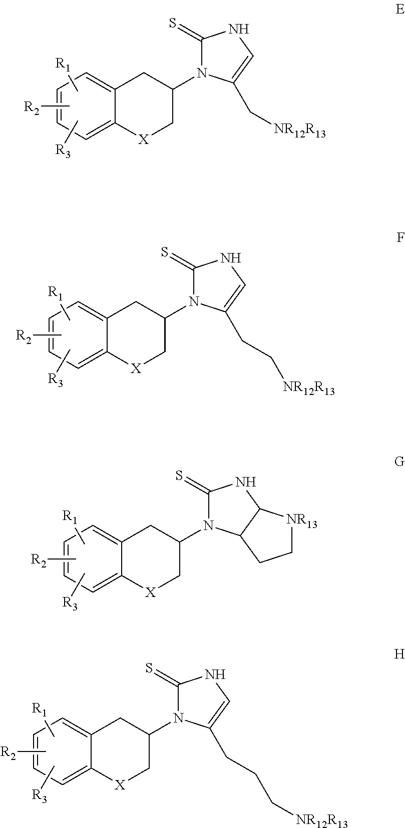

followed by subsequent deprotection of the intermediate products E to H to produce the respective R or S enantiomer of a compound of formula J or a salt thereof

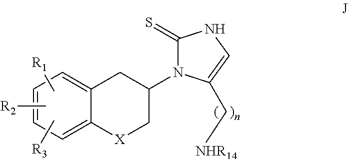

wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen, or nitro group; the term halogen means fluorine, chlorine, bromine or iodine.

In an embodiment, X is O. In another embodiment, n is 2 or 3. In an embodiment, X is O and n is 2. Alternatively, X is O and n is 3. In a further embodiment, at least one of R₁, R₂, and R₃ is fluorine. Optionally, the compound of formula J is: (S)-5-(2-aminoethyl)-1-(1,2,3,4-tetrahydronaphthalen-2-yl)-1,3-dihydroimidazole-2-thione; (S)-5-(2-aminoethyl)-1-(5,7-difluoro-1,2,3,4-tetrahydronaphthalen-2-yl)-1,3-dihydroimidazole-2-thione; (R)-5-(2-aminoethyl)-1-chroman-3-yl-1,3-dihydroimidazole-2-thione; (R)-5-(2-aminoethyl)-1-(6-hydroxychroman-3-yl)-1,3-dihydroimidazole-2-thione; (R)-5-(2-aminoethyl)-1-(8-hydroxychroman-3-yl)-1,3-dihydroimidazole-2-thione; (R)-5-(2-aminoethyl)-1-(6-methoxychroman-3-yl)-1,3-dihydroimidazole-2-thione; (R)-5-(2-aminoethyl)-1-(8-methoxychroman-3-yl)-1,3-dihydroimidazole-2-thione; (R)-5-(2-aminoethyl)-1-(6-fluorochroman-3-yl)-1,3-dihydroimidazole-2-thione; (R)-5-(2-aminoethyl)-1-(8-fluorochroman-3-yl)-1,3-dihydroimidazole-2-thione; (R)-5-(2-aminoethyl)-1-(6,7-difluorochroman-3-yl)-1,3-dihydroimidazole-2-thione; (R)-5-(2-aminoethyl)-1-(6,8-difluorochroman-3-yl)-1,3-dihydroimidazole-2-thione; (S)-5-(2-aminoethyl)-1-(6,8-difluorochroman-3-yl)-1,3-dihydroimidazole-2-thione; (R)-5-(2-aminoethyl)-1-(6,7,8-trifluorochroman-3-yl)-1,3-dihydroimidazole-2-thione; (R)-5-(2-aminoethyl)-1-(6-chloro-8-methoxychroman-3-yl)-1,3-dihydroimidazole-2-thione; (R)-5-(2-amino ethyl)-1-(6-methoxy-8-chlorochroman-3-yl)-1,3-dihydroimidazole-2-thione; (R)-5-(2-amino ethyl)-1-(6-nitro chroman-3-yl)-1,3-dihydroimidazole-2-thione; (R)-5-(2-amino ethyl)-1-(8-nitro chroman-3-yl)-1,3-dihydroimidazole-2-thione; (R)-5-(2-amino ethyl)-1-[6-(acetylamino)chroman-3-yl]-1,3-dihydroimidazole-2-thione; (R)-5-aminomethyl-1-chroman-3-yl-1,3-dihydroimidazole-2-thione; (R)-5-aminomethyl-1-(6-hydroxychroman-3-yl)-1,3-dihydroimidazole-2-thione; (R)-5-(2-amino ethyl)-1-(6-hydroxy-7-benzylchroman-3-yl)-1,3-dihydroimidazole-2-thione; (R)-5-aminomethyl-1-(6,8-difluorochroman-3-yl)-1,3-dihydroimidazole-2-thione; (R)-5-(3-aminopropyl)-1-(6,8-difluoro chroman-3-yl)-1,3-dihydroimidazole-2-thione; (S)-5-(3-aminopropyl)-1-(5,7-difluoro-1,2,3,4-tetrahydronaphthalen-2-yl)-1,3-dihydroimidazole-2-thione; (R,S)-5-(2-amino ethyl)-1-(6-hydroxythiochroman-3-yl)-1,3-dihydroimidazole-2-thione; (R,S)-5-(2-amino ethyl)-1-(6-methoxythiochroman-3-yl)-1,3-dihydroimidazole-2-thione; (R)-5-(2-b enzylamino ethyl)-1-(6-methoxychroman-3-yl)-1,3-dihydroimidazole-2-thione; (R)-5-(2-b enzylamino ethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluoro chroman-3-yl)-5-(2-methylaminoethyl)-1,3-dihydroimidazole-2-thione or (R)-1-chroman-3-yl-5-(2-methylaminoethyl)-1,3-dihydroimidazole-2-thione.

The compound of formula J may also be a salt of: (S)-5-(2-amino ethyl)-1-(1,2,3,4-tetrahydronaphthalen-2-yl)-1,3-dihydroimidazole-2-thione; (S)-5-(2-amino ethyl)-1-(5,7-difluoro-1,2,3,4-tetrahydronaphthalen-2-yl)-1,3-dihydroimidazole-2-thione; (R)-5-(2-amino ethyl)-1-chroman-3-yl-1,3-dihydroimidazole-2-thione; (R)-5-(2-amino ethyl)-1-(6-hydroxychroman-3-yl)-1,3-dihydroimidazole-2-thione; (R)-5-(2-amino ethyl)-1-(8-hydroxychroman-3-yl)-1,3-dihydroimidazole-2-thione; (R)-5-(2-amino ethyl)-1-(6-methoxychroman-3-yl)-1,3-dihydroimidazole-2-thione; (R)-5-(2-amino ethyl)-1-(8-methoxychroman-3-yl)-1,3-dihydroimidazole-2-thione; (R)-5-(2-amino ethyl)-1-(6-fluorochroman-3-yl)-1,3-dihydroimidazole-2-thione; (R)-5-(2-amino ethyl)-1-(8-fluorochroman-3-yl)-1,3-dihydroimidazole-2-thione; (R)-5-(2-amino ethyl)-1-(6,7-difluorochroman-3-yl)-1,3-dihydroimidazole-2-thione; (R)-5-(2-amino ethyl)-1-(6,8-difluorochroman-3-yl)-1,3-dihydroimidazole-2-thione; (S)-5-(2-amino ethyl)-1-(6,8-difluorochroman-3-yl)-1,3-dihydroimidazole-2-thione; (R)-5-(2-amino ethyl)-1-(6,7,8-trifluorochroman-3-yl)-1,3-dihydroimidazole-2-thione; (R)-5-(2-amino ethyl)-1-(6-chloro-8-methoxychroman-3-yl)-1,3-dihydroimidazole-2-thione; (R)-5-(2-amino ethyl)-1-(6-methoxy-8-chlorochroman-3-yl)-1,3-dihydroimidazole-2-thione; (R)-5-(2-amino ethyl)-1-(6-nitrochroman-3-yl)-1,3-dihydroimidazole-2-thione; (R)-5-(2-amino ethyl)-1-(8-nitrochroman-3-yl)-1,3-dihydroimidazole-2-thione; (R)-5-(2-amino ethyl)-1-[6-(acetylamino)chroman-3-yl]-1,3-dihydroimidazole-2-thione; (R)-5-aminomethyl-1-chroman-3-yl-1,3-dihydroimidazole-2-thione; (R)-5-aminomethyl-1-(6-hydroxychroman-3-yl)-1,3-dihydroimidazole-2-thione; (R)-5-(2-amino ethyl)-1-(6-hydroxy-7-benzylchroman-3-yl)-1,3-dihydroimidazole-2-thione; (R)-5-aminomethyl-1-(6,8-difluorochroman-3-yl)-1,3-dihydroimidazole-2-thione; (R)-5-(3-aminopropyl)-1-(6,8-difluorochroman-3-yl)-1,3-dihydroimidazole-2-thione; (S)-5-(3-aminopropyl)-1-(5,7-difluoro-1,2,3,4-tetrahydronaphthalen-2-yl)-1,3-dihydroimidazole-2-thione; (R,S)-5-(2-amino ethyl)-1-(6-hydroxythiochroman-3-yl)-1,3-dihydroimidazole-2-thione; (R,S)-5-(2-amino ethyl)-1-(6-methoxythiochroman-3-yl)-1,3-dihydroimidazole-2-thione; (R)-5-(2-b enzylamino ethyl)-1-(6-methoxychroman-3-yl)-1,3-dihydroimidazole-2-thione; (R)-5-(2-b enzylamino ethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluoro chroman-3-yl)-5-(2-methylaminoethyl)-1,3-dihydroimidazole-2-thione or (R)-1-chroman-3-yl-5-(2-methylaminoethyl)-1,3-dihydroimidazole-2-thione.

Preferably, the salt is the hydrochloride salt.

In an embodiment, the compound of formula J is the respective R or S enantiomer of the compound of formula 1:

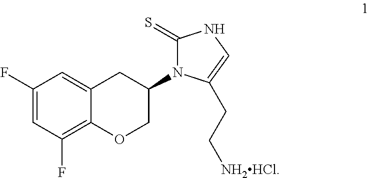

According to another aspect of the present invention, there is provided the use of a transition metal complex comprising a chiral catASium™ T series ligand having the formula:

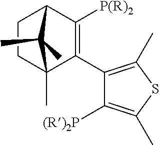

wherein R and R′ are as described above, in the asymmetric hydrogenation of a compound of formula B,

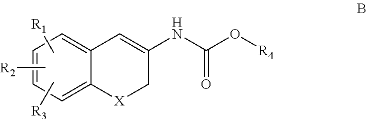

wherein compound B is as described above.

Preferably, the catalyst is Ru(catASium™ T series ligand)(acac)₂, Ru(catASium™ T series ligand)Br₂, Ru(catASium™ T series ligand)Cl₂(Ar) wherein Ar is C₆H₆ or p-cymene, or Ru(catASium™ T series ligand)Cl₂(dmf)ₓ, wherein x is suitably 2, 3, or 4. Preferably, the catalyst has the formula Ru(catASium™ T series ligand)(acac)₂.

Preferably, the catASium™ T series ligand is the R or S enantiomer of catASium™ T1, catASium™ T2, catASium™ T3, or catASium™ T4. Preferably, the catASium™ ligand is in the form of the R enantiomer. Most preferably, the catASium™ T series ligand is the R enantiomer of catASium™ T3. The most preferred catalyst has the formula Ru(catASium™ T3)(acac)₂.

In an embodiment, the catalyst is pre-formed.

In another embodiment, the hydrogenation is carried out in the presence of an acid.

According to another aspect of the present invention, there is provided a process for preparing a pre-formed transition metal catalyst comprising a catASium™ T ligand of the following formula:

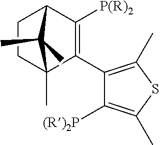

wherein R and R′ have the same meanings as defined above, the process comprising reacting a transition metal pre-cursor compound of [Ru(C₆H₆)Cl₂]₂ with the catASium™ T ligand in DMF and isolating the transition metal catalyst before the catalyst is used in a subsequent process. The catalyst may be Ru(catASium™ T series ligand)Cl₂(dmf)ₓ wherein x is 2, 3, or 4.

According to another aspect of the present invention, there is provided a process for preparing a transition metal catalyst comprising a catASium™ T ligand of the following formula

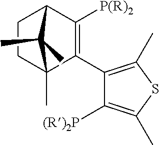

wherein R and R′ have the same meanings as defined above, the process comprising reacting a transition metal pre-cursor compound with the catASium™ T ligand, wherein the pre-cursor compound is not [Ru(C₆H₆)Cl₂]₂ and the solvent is not DMF.

In an embodiment, the transition metal catalyst is isolated before being used in a subsequent process. In an alternative embodiment, the transition metal catalyst is formed in situ.

In an embodiment, the catalyst is Ru(catASium™ T series ligand)(acac)₂, Ru(catASium™ T series ligand)Br₂ or Ru(catASium™ T series ligand)Cl₂(C₆H₆).

In an embodiment, the catalyst is Ru(catASium™ T ligand)(acac)₂ catalyst and the pre-cursor is Ru(η⁴-hexadiene)(acac)₂.

In an embodiment, the catalyst is Ru(catASium™ T ligand)Br₂ and the pre-cursor is Ru(methylallyl)₂COD.

In an embodiment, the catalyst is Ru(catASium™ T series ligand)Cl₂(C₆H₆), the pre-cursor is [Ru(C₆H₆)Cl₂]₂, and the process is carried out in the presence of a 1:1 mixture of dichloromethane/ethanol.

In an embodiment, the catalyst is Ru(catASium™ T series ligand)Cl₂(p-cymene), the pre-cursor is [Ru(p-cymene)Cl₂]₂, and the process is carried out in the presence of a 1:1 mixture of dichloromethane/ethanol.

Suitable catASium™ T series ligands are shown above in Scheme 1. Preferred catASium™ T series ligands are the R or S enantiomer of catASium™ T3, more preferably the R enantiomer of catASium™ T3.

According to another aspect of the present invention, there is provided a process for preparing the S or R enantiomer of a compound of formula A according to the process described above, wherein the chiral transition metal catalyst is prepared according to the process described above.

In an embodiment, the chiral transition metal catalyst is isolated before being reacted with the compound of formula B.

In an embodiment, the chiral transition metal catalyst is formed in situ. In other words, the catalyst is not isolated before being reacted with the compound of formula B.

According to another aspect of the present invention, there is provided Ru(catASium™ T ligand)(acac)₂, wherein the catASium™ T ligand is the R or S enantiomer of catASium™ T3, preferably the R enantiomer of catASium™ T3, and may be produced according to the process described above. In an embodiment, the Ru(catASium™ T ligand)(acac)₂ is in isolation. In an embodiment, the Ru(catASium™ T ligand)(acac)₂ is prepared according to the process described above.

According to another aspect of the present invention, there is provided Ru(catASium™ T ligand)Br₂, wherein the catASium™ T ligand is the R or S enantiomer of catASium™ T3, preferably the R enantiomer of catASium™ T3, and may be produced according to the process described above. In an embodiment, the Ru(catASium™ T ligand)Br₂, is in isolation. In an embodiment, the Ru(catASium™ T ligand)Br₂ is prepared according to the process described above.

According to another aspect of the present invention, there is provided Ru(catASium™ T ligand)Cl₂(dmf)ₓ in isolation, wherein x is 2, 3, or 4 and the catASium™ T ligand is the R or S enantiomer of catASium™ T3, preferably the R enantiomer of catASium™ T3, and may be produced according to the process described above. In an embodiment, the Ru(catASium™ T ligand)Cl₂(dmf)ₓ is prepared according to the process described above.

According to another aspect of the present invention, there is provided Ru(catASium™ T ligand)Cl₂(C₆H₆), wherein the catASium™ T ligand is the R or S enantiomer of catASium™ T3, preferably the R enantiomer of catASium™ T3, and may be produced according to the process described above. In an embodiment, the Ru(catASium™ T ligand)Cl₂(C₆H₆) is in isolation. In another embodiment, the Ru(catASium™ T ligand)Cl₂(C₆H₆) is prepared according to the process described above.

According to another aspect of the present invention, there is provided Ru(catASium™ T ligand)Cl₂(p-cymene), wherein the catASium™ T ligand is the R or S enantiomer of catASium™ T3, preferably the R enantiomer of catASium™ T3, and may be produced according to the process described above. In an embodiment, the Ru(catASium™ T ligand)Cl₂(p-cymene) is in isolation. In another embodiment, the Ru(catASium™ T ligand)Cl₂(p-cymene) is prepared according to the process described above.

According to another aspect of the present invention, there is provided (R)-5-(2-amino ethyl)-1-(6,8-difluoro chroman-3-yl)-1,3-dihydroimidazole-2-thione hydrochloride produced by a process described above.

### EXAMPLES

An investigation of the effect of the catalyst on the enantioselective hydrogenation of the prochiral methyl ene-carbamate 1d (as shown in Scheme 2 below) was carried out using ruthenium-catASium™ T-based catalysts (Tables 1 to 3 and 5 to 11) and rhodium-catASium™ T-based catalysts (Table 4).

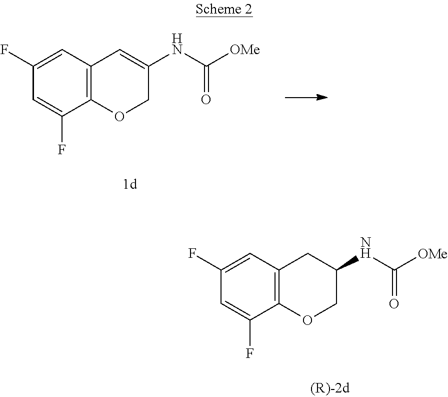

### Ruthenium-catASium™ T Catalysis

Ruthenium-based catalysis was carried out in the presence and absence of phosphoric acid.

The catalytically active Ru complexes were pre-formed before addition of the substrate: Ru(ligand)Cl₂(dmf)ₓ from [Ru(C₆H₆)Cl₂]₂ and ligand in DMF; [Ru(ligand)(Ar)Cl]Cl from [Ru(Ar)Cl₂]₂ and ligand in ethanol-dichloromethane 1:1 mixture, where Ar is C₆H₆ or p-cymene; [Ru(ligand)(acac)₂] from [Ru(η⁴-2,4-C₆H₁₀)(acac)₂] and ligand in dichloromethane; [RuBr₂(ligand)] from Ru(2-methylallyl)₂COD, ligand and HBr. The experimental conditions for these pre-formations are given below.

MPC 1: Pre-Formation of Ru(Ligand)Cl₂(dmf)ₓ,

0.001 mmol of each ligand and 0.0005 mmol of [Ru(C₆H₆)Cl₂]₂ were dissolved under argon in 0.05 ml DMF and warmed at 105° C. for 10 minutes. They were then cooled to room temperature.

### MPC 2: Pre-Formation of Ru(Ligand)Cl₂(C₆H₆).

0.001 mmol of each ligand and 0.0005 mmol of [Ru(C₆H₆)Cl₂]₂ were dissolved under argon in 0.1 ml of a mixture 1:1 dichloromethane/ethanol and warmed to 50° C. for 1.5 hours. They were then cooled to room temperature.

### MPC 3: Pre-Formation of Ru(Ligand)(acac)₂

The synthesis of this ruthenium salt was taken from Ziegler, M. L., et al., Organometallics 1991, 10, 3635-3642. The activation of zinc was carried out according to Knochel, P., et al., in “Preparation of highly functionalised reagents” in Organocopper Reagents, Oxford University Press, Oxford 1994, p. 85.

0.001 mmol of each ligand and 0.001 mmol of Ru(η⁴-hexadien)(acac)₂ were dissolved under argon in 0.1 ml dichloromethane and stirred at room temperature for 20-30 minutes.

### MPC 4: Pre-Formation of Ru(Ligand)Br₂

0.001 mmol of each ligand and 0.001 mmol of Ru(methylallyl)₂COD were dissolved under argon in 0.05 ml acetone and 2 equivalents of HBr (solution made from aqueous 48% HBr diluted in methanol) were added. The mixture was stirred for 30 minutes at room temperature.

### Hydrogenation Conditions

Reproducibility experiments were performed in MeOH at 60° C. and 30 bar H₂ for 18 hours at a S/C ratio of 100. More specifically, 0.4 ml of a 0.25M solution of substrate 1d in MeOH was added to the pre-formed ruthenium complexes and 50 μl of H₃PO₄ 85% was optionally added.

The reaction mixtures were then introduced into the autoclave and the autoclave was purged with hydrogen. Unless otherwise stated, 30 bar hydrogen was pressured and the reaction was warmed at 60° C. for 18 hours.

After cooling and releasing the pressure, a sample of the raw mixture (0.1 ml) was taken for analysis. The sample was diluted with MeOH, some Deloxan® was added to remove the metal from the reaction mixture and the mixture was shaken for 10 minutes at room temperature; after filtering through paper, the samples were diluted with 0.5 ml methanol and 0.5 ml iPrOH). An HPLC-method was established: Chiralpak AD, MeOH/iPrOH 70/30; 0.5 ml/min; 30° C.

Pre-Screening of catASium™ T2

The catASium™ T series ligand T2 was tested in the presence and the absence of phosphoric acid using the four Ruthenium-metal precursors described above (MPC1, MPC2, MPC3, and MPC4). A constant amount of phosphoric acid (50 μl) was added. The values of conversion (“Con”) and enantiomeric excess (“ee”) were confirmed twice for each catalyst.

The results of the experiments performed without and with phosphoric acid are summarised in Table 1 and Table 2, respectively.

TABLE 1
^{a}Pre-screening of catASium ™ T2 without H₃PO₄

| | | ee | Con | ee | Con | ee | Con | ee |
|---|---|---|---|---|---|---|---|---|
| | Con | MPC | MPC | MPC | MPC | MPC | MPC | MPC |
| | MPC 1 | 1 | 2 | 2 | 3 | 3 | 4 | 4 |
| catASium ™ | 100/ | 87/68 | 100/ | 79/79 | 8/8 | 56/46 | 100/ | 87/86 |
| T2 | 100 | | 100 | | | | 100 | |

^{a}Conversions (“Con”) and ee are given in %. In each entry are given the two values of the two confirmations.

TABLE 2
^{a}Pre-screening of catASium ™ T2 in the presence of H₃PO₄^{b}

| | Con | ee | Con | ee | Con | ee | Con | ee |
|---|---|---|---|---|---|---|---|---|
| | MPC | MPC | MPC | MPC | MPC | MPC | MPC | MPC |
| | 1 | 1 | 2 | 2 | 3 | 3 | 4 | 4 |
| catASium ™ | 100/ | 80/80 | 89/100 | 17/85 | 100/ | 93/93 | 100/ | 85/84 |
| T2 | 100 | | | | 100 | | 100 | |

^{a}Conversions (“Con”) and ee are given in %. In each entry are given the two values of the two confirmations.
^{b}50 μl of phosphoric acid was used. This means approximately 10% v/v.

Having demonstrated using catASium™ T2 that high conversions and selectivities could be reproduced and that the presence of phosphoric acid can have a beneficial effect on the catalyst performance, the catASium™ ligands T1 and T3 from the T series were investigated. The experimental conditions were the same as given above (in “Hydrogenation Conditions” section) and the results are summarised in Table 3 below.

TABLE 3
catASium ™ T1 and catASium ™ T3 Investigations^{a}

| Ligand | Metal precursor | Additive | ee (%) | By-products |
|---|---|---|---|---|
| catASium ™ T3 | MPC 2 | — | 90/89 | — |
| catASium ™ T1 | MPC 3 | H₃PO₄ | 94/94 | Traces of |
| | | | | ketone |
| catASium ™ T1 | MPC 4 | H₃PO₄ | 90/91 | — |
| catASium ™ T3 | MPC 3 | H₃PO₄ | 95/95 | Traces of |
| | | | | ketone |
| catASium ™ T3 | MPC 4 | H₃PO₄ | 92/93 | — |

^{a}The conversion was always 100%. The R-enantiomer was obtained. The ee-column shows the results of both confirmation experiments.

Complexes pre-formed from Ru (η⁴-hexadiene)(acac)₂ and the catASium™ T1, T2, and T3 ligands, when used in the presence of H₃PO₄, gave full conversion and 94% ee, 93% ee and 95% ee respectively.

Complexes pre-formed from Ru(methylallyl)₂(COD) and the catASium™ T1 and T3 ligands, when used in the presence of H₃PO₄, gave full conversion and over 90% ee (90-91% ee with T1 and 92-93% ee with T3).

### Rhodium-catASium™ T Catalysis

Hydrogenation of ene-carbamate 1d using catalysts of general formula [Rh(catASium™)(COD)]BF₄ in dichloromethane at 30° C., 30 bar H₂ led to low enantioselectivities (Table 4).

TABLE 4
^{a}Results obtained in the rhodium catalysed reactions

| | Con | ee | Con | ee | Con | ee | Con | ee |
|---|---|---|---|---|---|---|---|---|
| Ligand | MeOH | MeOH | THF | THF | DCM | DCM | Toluene | Toluene |
| catASium ™ T2 | 44/100 | 41/42 | 65/43 | 21/41 | 100/100 | 40/40 | 73/81 | 74/70 |
| catASium ™ T3 | 100/100 | 1/1 | 100/100 | 17/19 | 100/100 | 55/55 | 100/100 | 13/15 |

^{a}Conversions and ee are given in %. In each entry are given the two values of the two confirmations.

### Ruthenium-catASium™ T Catalysis Optimisation

### Solvent/Additive/Metal Precursor Optimisation

A substrate/catalyst (S/C) ratio of 250/1 was chosen. The pressure and the temperature were kept as in the previous experiments.

Other reaction parameters were chosen as follows:
The solvent: MeOH and iPrOH.
The additive: strong and weak acids were tested (5% H₃PO₄, 5% H₂SO₄, 5% HBr, 20% AcOH).
The metal precursor: Ru(η⁴-hexadien)(acac)₂ or Ru(methylallyl)₂COD were tested.

The experimental procedure was the same as above (in “Hydrogenation Conditions” section). The substrate was introduced as a 0.66M solution (0.4 ml) in the corresponding solvent. Because the additive was diluted in 0.4 ml of the solvent, the final substrate concentration was approx. 0.33M.

When using iPrOH as solvent it was observed that, in general, all reactions with high conversion presented as a main product the alcohol. In iPrOH the hydrolysis to the ketone and its reduction takes place preferentially to the hydrogenation of the ene-carbamate. Only one example was observed where no hydrolysis was observed. Thus, isopropanol was discarded and MeOH used. However, it may be that the use of iPrOH as a solvent at a lower acid concentration would result in suppression of the hydrolysis and preferential hydrogenation of ene-carbamate.

TABLE 5
Results obtained at S/C 250/1 in MeOH

| | Conv. | ee | Conv. | ee | Conv. | ee | Conv. | ee |
|---|---|---|---|---|---|---|---|---|
| Ligand | T1 | T1 | T2 | T2 | T3 | T3 | T4 | T4 |
| MPC 3 - H₃PO₄ | 100/100 | 92/93 | 100/100 | 92/92 | 100/100 | 94/94 | 100/100 | 92/93 |
| MPC 3 - AcOH | 81/81 | 93/95 | 85/96 | 92/92 | 99/99 | 95/94 | 100/100 | 91/91 |
| MPC 4 - H₃PO₄ | 92/90 | 85/85 | 100/100 | 83/82 | 100/100 | 88/87 | 100/100 | 89/89 |
| MPC 4 - AcOH | 35/36 | 56/58 | 61/61 | 68/69 | 66/70 | 73/76 | 95/83 | 80/82 |

Table 6 summarises the best results from Table 5 (conversion >96%; ee>90%)

TABLE 6
Summary of the best results in MeOH at S/C 250/1^{a}

| Ligand | Additive | ee (%) | By-products |
|---|---|---|---|
| catASium T1 | H₃PO₄ | 92/93 | Traces of alcohol and ketone |
| catASium T2 | H₃PO₄ | 92/92 | Traces of alcohol |
| catASium T3 | H₃PO₄ | 94/94 | Traces of alcohol and ketone |
| catASium T3^{b} | AcOH | 95/94 | — |
| catASium T4 | H₃PO₄ | 92/93 | Traces of alcohol |
| catASium T4 | AcOH | 91/91 | — |

^{a}Metal precursor: MPC 3; except where indicated otherwise the conversion was 100%. The R-enantiomer was obtained. The ee-column shows the results of both confirmation experiments.
^{b}Conversion: 99%.

### Temperature/Pressure/Concentration of Additive Optimisation

Temperature (50° C., 60° C., and 80° C.), pressure (20, 30, and 70 bar hydrogen), and concentration of acidic additive were varied at a more demanding S/C ratio (500/1). At this point it was decided to proceed with MPC 3 (all results in Table 6 were obtained with MPC 3).

The experimental procedure was the same as above (in “Hydrogenation Conditions” section). The substrate was introduced as a 0.66M solution (0.8 ml) in the corresponding solvent. Because the additive was diluted in 0.8 ml of the corresponding solvent the final substrate concentration was approx. 0.33M. The reactions were performed at the pressure and temperature values given in the tables.

The best results from each experiment have been grouped by ligand:
The results obtained with catASium™ T1 are summarised in Table 7.
The results obtained with catASium™ T2 are summarised in Table 8.
The results obtained with catASium™ T3 are summarised in Table 9.

TABLE 7
Results obtained with catASium ™ T1

| Conversion/ | T = 60° C. | | | T = 80° C. | | |
|---|---|---|---|---|---|---|
| ee | 20 bar | 30 bar | 70 bar | 20 bar | 30 bar | 70 bar |
| 0.01% H₃PO₄ | | 0/0 | 30/89 | | 38/89 | 100/91 |
| 0.1% H₃PO₄ | 25/91 | 12/87 | 46/91 | 92/93 | 100/92 | 100/91 |
| 1% H₃PO₄ | 0/0 | | | 93/92 | | |
| No acid | | 0/0 | 2/57 | | 0/0 | 72/87 |

This ligand (T1) works well at high temperatures and pressures.
The presence of the acid aids in obtaining high conversions.
When using 0.01% H₃PO₄ the reaction works better at 80° C. and 70 bar; when using 0.1% H₃PO₄ the reaction works well at 30 bar as well as at higher pressures.

TABLE 8
Results obtained with catASium ™ T2

| Conversion/ | T = 60 | | | T = 80 | | |
|---|---|---|---|---|---|---|
| ee | 20 bar | 30 bar | 70 bar | 20 bar | 30 bar | 70 bar |
| 0.01% H₃PO₄ | | 0/0 | 67/90 | | 28/87 | 100/90 |
| 0.1% H₃PO₄ | 41/91 | 0/0 | 72/90 | 100/92 | 0/0 | 100/90 |
| 1% H₃PO₄ | 0/0 | | | 14/65 | | |
| No acid | | 0/0 | 3/60 | | 0/0 | 100/86 |

The behaviour of this ligand is similar to catASium™ T1:
This ligand (T2) works well at high temperatures and pressures.
The presence of the acid aids in obtaining high conversions.
When using 0.01% H₃PO₄ the reaction works better at 80° C. and 70 bar; when using 0.1% H₃PO₄ the reaction works well at 30 bar as well as at higher pressures.

TABLE 9
Results obtained with catASium ™ T3

| Conversion/ | T = 60 | | | T = 80 | | |
|---|---|---|---|---|---|---|
| ee | 20 bar | 30 bar | 70 bar | 20 bar | 30 bar | 70 bar |
| 0.01% H₃PO₄ | | 18/86 | 50/92 | | 91/93 | 100/92 |
| 0.1% H₃PO₄ | 43/94 | 37/79 | 73/94 | 100/95 | 100/93 | 100/92 |
| 1% H₃PO₄ | 0/0 | | | 100/94 | | |
| 25% Acetic | 3/88 | 0/0 | 96/89 | 0/0 | 0/0 | 100/93 |
| acid | | | | | | |
| 50% Acetic | 7/96 | 0/0 | 100/93 | 5/91 | 0/0 | 100/90 |
| acid | | | | | | |
| No acid | | 0/0 | 5/62 | | 26/88 | 100/89 |
| 0.005% H₃PO₄ | | 0/0 | 33/90 | | 100/94 | 100/91 |

This ligand presented the best reactivity:
The presence of the acid is preferable for obtaining high conversions. The acid can be avoided by working at high temperature and high pressures.
By increasing the temperature, good reactivity was observed even at 20 bar. At high temperatures and low pressures only 0.1% H₃PO₄ is necessary for 100% conversion and 95% ee.
The best results (in conversion) are obtained at high temperature and pressure. However, the enantiomeric excess is some units lower. By using high temperature and pressure no acid is necessary.

### S/C Optimisation

The optimization of the S/C was carried out with the best system (catASium™ T3). Different S/C (1000, 2000, 4000, 5000) ratios were tested with catASium™ T3 at 30 bar and 80° C. in the presence of 0.1% H₃PO₄. There are two ways for increasing the S/C ratio:
by keeping constant the amount of substrate (maintaining constant the concentration at the same values as in the experiments at S/C 500) and lowering the amount of catalyst.
by keeping constant the amount of catalyst and increasing the amount of substrate.

Both ways were tested. The two experiments were carried out in the presence of 1% phosphoric acid.

The experimental procedure was as above in “Hydrogenation Conditions” Section.

The substrate was weighed for each test and the corresponding amount of methanol was added. The concentrations are summarised in Table 10 and the results are summarised in Table 11. The reactions were performed at an initial pressure of 30 bar hydrogen and at 80° C. temperature.

TABLE 10
Reaction conditions

| | Substrate constant | | Catalyst constant | |
|---|---|---|---|---|
| | mmol | | mmol | |
| | Substrate/ | | Substrate/ | |
| 0.1% H₃PO₄ | μmol | MeOH (ml)/ | μmol | MeOH (ml)/ |
| S/C | Catalyst | [c]_{Substrate} (M) | Catalyst | [c]_{Substrate} (M) |
| 1000 | 1/1 | 3/0.33 | 1/1 | 3/0.33 |
| 2000 | 1/0.5 | 3/0.33 | 2/1 | 3/0.66 |
| 4000 | 1/0.25 | 3/0.33 | 4/1 | 3/1.33 |
| 5000 | 1/0.2 | 3/0.33 | 5/1 | 3/1.66 |

TABLE 11
Results obtained at high S/C ratios (0.1% H₃PO₄)

| | | |
|---|---|---|
| 0.1% H₃PO₄ | Substrate constant | Catalyst constant |

| S/C | Conversion | ee | Conversion | ee |
|---|---|---|---|---|
| 1000 | 100/100 | 91/92 | 98/99 | 91/86 |
| 2000 | 19/10 | 84/84 | 0/0 | 0/0 |
| 4000 | 0/3 | 0/76 | 0/5 | 0/5 |
| 5000 | 0/0 | 0/0 | 48 | 2 |

TABLE 11
(continuation) Results obtained at high S/C ratios (1% H₃PO₄)

| | |
|---|---|
| 1% H₃PO₄ | Catalyst constant |

| S/C | Conversion | ee |
|---|---|---|
| 1000 | 75/71 | 74/79 |

The differences in conversion indicate that stirring the reaction mixture could aid in achieving good conversion.

### Enantiopurity Upgrade

The enantiomeric excess may be increased by crystallisation of the crude product. For example, the crystallisation may involve evaporated any residual solvent from the crude product, dissolving the residue in the minimal amount of warmed dichloromethane. After filtering, adding hexane slowly until the product began to crystallise. After crystallising for 3 hours at room temperature and 15 hours at 4° C., the crystals were filtered and washed with hexane.

### Scale-Up Experiment

In order to investigate the effectiveness of the catalyst on a large scale, the following reaction was carried out on an 800 g scale (in a 15 L autoclave):

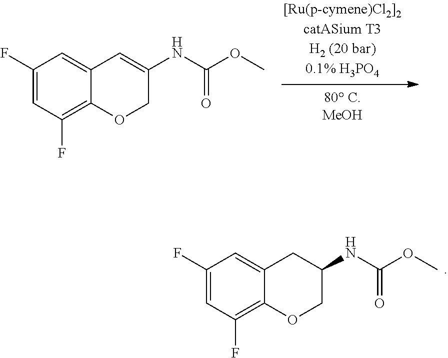

The experimental procedure was as follows:

Catalyst: [Ru(p-cymene)Cl₂]₂/catASium™ T3 in EtOH/CH₂Cl₂

### Pressure: 20 bar

### Temperature: 80° C.

### S/C: 2000

### Concentration: 0.7 M

### Additive: 0.1% H₃PO₄.

[Ru(p-cymene)Cl₂]₂ and catASium™ T3 were stirred at 50° C. for 90 minutes in a mixture of dichloromethane/EtOH (1:1) and then cooled to room temperature. The 15 L autoclave was charged with the substrate, methanol and the corresponding additive under argon atmosphere. Afterwards the catalyst was added. The reaction was hydrogenated for 18 hours at the conditions given above.

Deloxan® was added to the reaction mixture and the catalyst was separated by filtration. During the evaporation of the solvent (approx. 2000 ml out of 6000 ml), a formation of a precipitation occurred. The distillation was stopped at approx. 5000 ml of distillate and the precipitation was filtered off and washed with a small amount of methanol. The isolated solid (white crystals) was dried under vacuum (180-210 mbar) at 40° C. for 18 hours. The filtrate was evaporated to dryness to obtain a green-brown solid.

The results are shown in Table 12.

TABLE 12
Results of the 800 g scale experiment

| | Conversion | ee | Product | | | |
|---|---|---|---|---|---|---|
| Entry | [%] | [%] | (isolated) | | Yield | Comments |
| 1 | >99¹ | 95 | — | | — | reaction mixture |
| | | | | | | after 18 h |
| 2 | >99¹ | >99 | 730.43 | g | 90.55% | precipitation during |
| | | | | | | the evaporation of |
| | | | | | | the solvent |
| 3 | >99¹ | 26 | 71 | g | 8.80% | filtrate (mother |
| | | | | | | liquor; solvent free) |

¹starting material was not detected via HPLC

Thus, it has been found that with 800 g substrate and a substrate/catalyst ratio of 2000:1, the desired chiral product was produced with optical purity greater than 99% and at a yield of 91%.

It will be appreciated that the invention may be modified within the scope of the appended claims.

1. A process for preparing the S or R enantiomer of a compound of formula A, 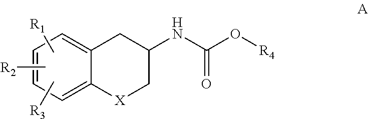
the process comprising subjecting a compound of formula B to asymmetric hydrogenation in the presence of a chiral transition metal catalyst and a source of hydrogen, 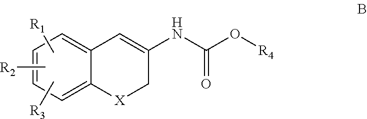
wherein X is CH₂, oxygen or sulphur; R₁, R₂ and R₃ are the same or different and signify hydrogen, halogen, alkyl, alkyloxy, hydroxy, nitro, alkylcarbonylamino, alkylamino or dialkylamino group; and R₄ is alkyl or aryl, the transition metal catalyst comprising a chiral ligand having the formula 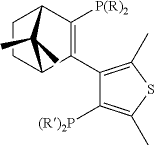
wherein each R and R′ independently represents alkyl, aryl, aralkyl, alkenyl, alkynyl, alkoxy, aryloxy, alkylthio, arylthio, unsubstituted or substituted cyclic moiety selected from a group consisting of monocyclic or polycyclic saturated or partially saturated carbocyclic or heterocyclic, or aromatic or heteroaromatic rings, said rings comprising from 4 to 8 atoms and comprising from 0 to 3 heteroatoms, wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means an aromatic or heteraromatic group, optionally substituted one or more times by alkyl, alkyloxy, halogen or nitro group; and the term halogen means fluorine, chlorine, bromine or iodine.

2. The process according to claim 1, wherein X is O.

3. The process according to claim 1, wherein at least one of R₁, R₂ and R₃ is fluorine.

4. The process according to claim 1, wherein compound A has the following formula:

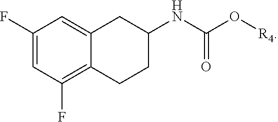

5. The process according to claim 1, wherein R₄ is C₁ to C₄ alkyl, preferably R₄ is methyl, ethyl or ^{t}Bu, more preferably R₄ is methyl.

6-7. (canceled)

8. The process according to claim 1, wherein R₄ is benzyl.

9. The process according to claim 1 wherein the catalyst has the formula [(chiral ligand)Ru(arene)X′]Y, [(chiral ligand)Ru(L)₂] or [(chiral ligand)Ru(L′)₂X′₂], wherein X′ is a singly-negative monodentate ligand, Y is a balancing anion, L is a monovalent negative coordinating ligand and L′ is a non-ionic monodentate ligand.

10-13. (canceled)

14. The process according to claim 1 wherein the catalyst is Ru(chiral ligand)(acac)₂.

15. (canceled)

16. The process according to claim 1, wherein the catalyst is Ru(chiral ligand)Br₂.

17. (canceled)

18. The process according to claim 1 wherein the catalyst is Ru(chiral ligand)Cl₂(dmf)ₓ, wherein x is 2, 3 or 4.

19. (canceled)

20. The process according to claim 1, wherein the catalyst is Ru(chiral ligand)Cl₂(C₆H₆).

21. (canceled)

22. The process according to claim 1, wherein the chiral ligand is the R or the S enantiomer of a compound having one of the following structures:

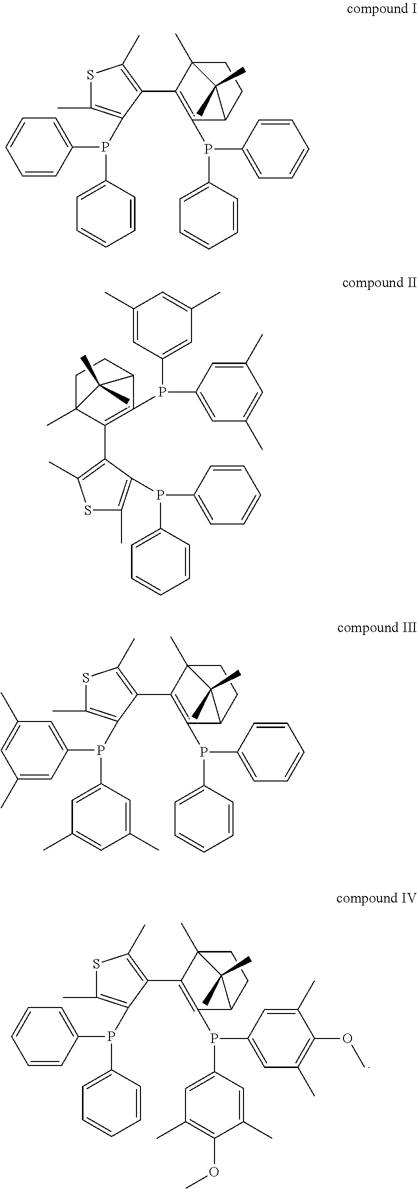

23-24. (canceled)

25. The process according to claim 1, wherein the hydrogenation is carried out in the presence of an acid, wherein the acid is CH₃COOH or H₁PO₄.

26-28. (canceled)

29. The process according to claim 1, wherein the hydrogenation is carried out in the presence of a solvent selected from MeOH, EtOH, 1-BuOH, 2-BuOH, CF₃CH₂OH, DCM, DCE, THF, toluene or a 1:1 mixture of MeOH, toluol and DCM.

30-33. (canceled)

34. The process according claim 1, wherein the hydrogenation is carried out at a temperature ranging from 40° C. to 100° C.

35-38. (canceled)

39. The process according to claim 1, wherein the hydrogenation is carried out at a pressure ranging from 10 bars to 70 bars.

40-44. (canceled)

45. The process according to claim 1, wherein the substrate:catalyst (S/C) ratio ranges from 100/1 to 5000/1.

46-48. (canceled)

49. The process according to claim 1, further comprising subsequently crystallising the compound of formula A.

50-52. (canceled)

53. A process for preparing the R or S enantiomer of a compound of formula C, 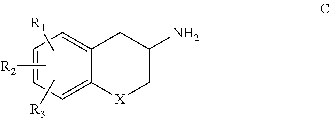
comprising forming the R or S enantiomer of a compound of formula A by a process according to claim 1, followed by converting the R or S enantiomer of the compound A to the respective R or S enantiomer of the compound of formula C.

54-55. (canceled)

56. A process for preparing the R or S enantiomer of a compound of formula E or a salt thereof: 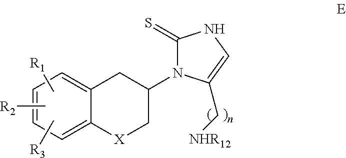
comprising forming the R or S enantiomer of a compound of formula C by a process according to claim 53, and converting the R or S enantiomer of the compound of formula C to the R or S enantiomer of the compound of formula E.

57-58. (canceled)

59. The process according to claim 56, comprising reacting the R or S enantiomer of the compound of formula C with a compound of formula D2 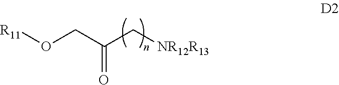
where n signifies 1, 2 or 3; when n is 1 or 2, R₁₂ signifies hydrogen, alkyl or alkylaryl group, R₁₁ signifies a hydroxyl protecting group and R₁₃ signifies an amino protecting group; when n signifies 3, R₁₁ signifies a hydroxyl protecting group but R₁₂ and R₁₃ taken together represent a phthalimido group; with a water soluble thiocyanate salt in the presence of an organic acid in a substantially inert solvent, followed by subsequent deprotection of the intermediate products F to I:

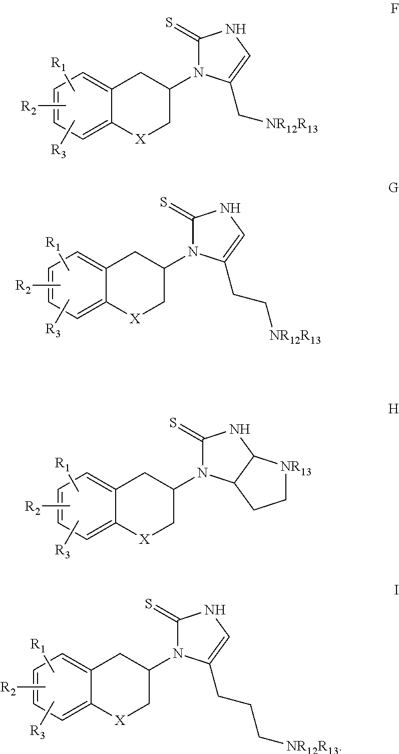

60-62. (canceled)

63. The process according to claim 56, wherein the compound E is (S)-5-(2-aminoethyl)-1-(1,2,3,4-tetrahydronaphthalen-2-yl)-1,3-dihydroimidazole-2-thione; (S)-5-(2-aminoethyl)-1-(5,7-difluoro-1,2,3,4-tetrahydronaphthalen-2-yl)-1,3-dihydroimidazole-2-thione; (R)-5-(2-aminoethyl)-1-chroman-3-yl-1,3-dihydroimidazole-2-thione; (R)-5-(2-aminoethyl)-1-(6-hydroxychroman-3-yl)-1,3-dihydroimidazole-2-thione; (R)-5-(2-aminoethyl)-1-(8-hydroxychroman-3-yl)-1,3-dihydroimidazole-2-thione; (R)-5-(2-aminoethyl)-1-(6-methoxychroman-3-yl)-1,3-dihydroimidazole-2-thione; (R)-5-(2-aminoethyl)-1-(8-methoxychroman-3-yl)-1,3-dihydroimidazole-2-thione; (R)-5-(2-aminoethyl)-1-(6-fluorochroman-3-yl)-1,3-dihydroimidazole-2-thione; (R)-5-(2-aminoethyl)-1-(8-fluorochroman-3-yl)-1,3-dihydroimidazole-2-thione; (R)-5-(2-aminoethyl)-1-(6,7-difluorochroman-3-yl)-1,3-dihydroimidazole-2-thione; (R)-5-(2-aminoethyl)-1-(6,8-difluorochroman-3-yl)-1,3-dihydroimidazole-2-thione; (S)-5-(2-aminoethyl)-1-(6,8-difluorochroman-3-yl)-1,3-dihydroimidazole-2-thione; (R)-5-(2-aminoethyl)-1-(6,7,8-trifluorochroman-3-yl)-1,3-dihydroimidazole-2-thione; (R)-5-(2-aminoethyl)-1-(6-chloro-8-methoxychroman-3-yl)-1,3-dihydroimidazole-2-thione; (R)-5-(2-aminoethyl)-1-(6-methoxy-8-chlorochroman-3-yl)-1,3-dihydroimidazole-2-thione; (R)-5-(2-aminoethyl)-1-(6-nitrochroman-3-yl)-1,3-dihydroimidazole-2-thione; (R)-5-(2-aminoethyl)-1-(8-nitrochroman-3-yl)-1,3-dihydroimidazole-2-thione; (R)-5-(2-aminoethyl)-1-[6-(acetylamino)chroman-3-yl]-1,3-dihydroimidazole-2-thione; (R)-5-aminomethyl-1-chroman-3-yl-1,3-dihydroimidazole-2-thione; (R)-5-aminomethyl-1-(6-hydroxychroman-3-yl)-1,3-dihydroimidazole-2-thione; (R)-5-(2-aminoethyl)-1-(6-hydroxy-7-benzylchroman-3-yl)-1,3-dihydroimidazole-2-thione; (R)-5-aminomethyl-1-(6,8-difluorochroman-3-yl)-1,3-dihydroimidazole-2-thione; (R)-5-(3-aminopropyl)-1-(6,8-difluorochroman-3-yl)-1,3-dihydroimidazole-2-thione; (S)-5-(3-aminopropyl)-1-(5,7-difluoro-1,2,3,4-tetrahydronaphthalen-2-yl)-1,3-dihydroimidazole-2-thione; (R,S)-5-(2-aminoethyl)-1-(6-hydroxythiochroman-3-yl)-1,3-dihydro imidazole-2-thione; at S)-5-(2-aminoethyl)-1-(6-methoxythiochroman-3-yl)-1,3-dihydroimidazole-2-thione; (R)-5-(2-benzylaminoethyl)-1-(6-methoxychroman-3-yl)-1,3-dihydroimidazole-2-thione; (R)-5-(2-benzylaminoethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluorochroman-3-yl)-5-(2-methylamino ethyl)-1,3-dihydroimidazole-2-thione or (R)-1-chroman-3-yl-5-(2-methylaminoethyl)-1,3-dihydroimidazole-2-thione, or a salt thereof.

64-65. (canceled)

66. The process according to claim 56, wherein the compound E is the respective R or S enantiomer of the compound of formula P:

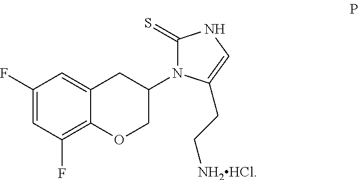

67. A method comprising utilizing a chiral transition metal catalyst in the asymmetric hydrogenation of a compound of formula B, 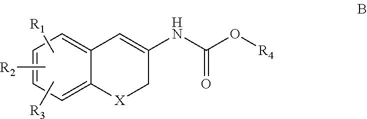
the transition metal catalyst comprising a chiral ligand having the formula 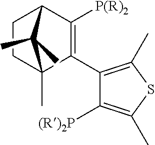
wherein each R and R′ independently represents alkyl, aryl, aralkyl, alkenyl, alkynyl, alkoxy, aryloxy, alkylthio, arylthio, unsubstituted or substituted cyclic moiety selected from a group consisting of monocyclic or polycyclic saturated or partially saturated carbocyclic or heterocyclic, or aromatic or heteroaromatic rings, said rings comprising from 4 to 8 atoms and comprising from 0 to 3 heteroatoms; X is CH₂, oxygen or sulphur; R₁, R₂ and R₃ are the same or different and signify hydrogen, halogen, alkyl, alkyloxy, hydroxy, nitro, alkylcarbonylamino, alkylamino or dialkylamino group; and R₄ is alkyl or aryl, wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means an aromatic or heteraromatic group, optionally substituted one or more times by alkyl, alkyloxy, halogen or nitro group; and the term halogen means fluorine, chlorine, bromine or iodine.

68. The method according to claim 67, wherein the catalyst is Ru(chiral ligand)(acac)₂, Ru(chiral ligand)Br₂, Ru(chiral ligand)Cl₂(dmf)ₓ wherein x is 2, 3 or 4, or Ru(chiral ligand)Cl₂(C₆H₆).

69-83. (canceled)
